(19) United States
(12) Patent Application Publication
(10) Pub. No.: US 2007/0129694 A1
(43) Pub. Date: Jun. 7, 2007

(54) Fluid evacuator system

(76) Inventors: John C. Opie, Scottsdale, AZ (US); Stephen J. Joyce, Phoenix, AZ (US); Thomas Izdebski, Phoenix, AZ (US)

Correspondence Address: Squire, Sanders & Dempsey L.L.P.;Suite 2700, Two Renaissance Squire, 40 North Central Avenue, Phoenix, AZ, 85004-4498, US

(21) Appl. No.: 11/600,597

(22) Filed: Nov. 14, 2006

Related U.S. Application Data
(63) Continuation of application No. 10/863,009, filed on Jun. 7, 2004.
(63) Continuation of application No. 10/559,400, filed on May 25, 2006.
(60) Provisional application No. 60/476,663, filed on Jun. 5, 2003.
(60) Provisional application No. 60/539,158, filed on Jan. 26, 2004.
(60) Provisional application No. 60/476,663, filed on Jun. 5, 2003.
(60) Provisional application No. 60/539,158, filed on Jan. 26, 2004.

(51) Int. Cl. A61M 1/00

(52) U.S. Cl. 604/313; 604/319

(57) ABSTRACT

A device is delineated for removing body fluid from a wound site. The device may include a drain sealed within the wound site, a conduit connected to the drain and a fluid container connected to the other end of the conduit. The fluid container may include at least one inlet passage of larger diameter to reduce likelihood of clogging during body-fluid inflow. An additional inlet passage may be included. The fluid container may also include at least one outlet passage of larger diameter to improve draining efficiency.

### RELATED APPLICATIONS

This application is a continuation of and claims priority to U.S. patent application Ser. No. 10/863,009, filed Jun. 7, 2004, which claims the benefit of U.S. Provisional Application Ser. No. 60/476,663, filed Jun. 5, 2003 and U.S. Provisional Application Ser. No. 60/539,158, filed Jan. 26, 2004, and is a continuation of U.S. patent application Ser. No. 10/559,400, filed May 25, 2006, which is the National Stage of International Application No. PCT/US04/18134, filed on Jun. 7, 2004, which claims the benefit of U.S. Provisional Application Ser. No. 60/476,663, filed Jun. 5, 2003 and U.S. Provisional Application Ser. No. 60/539,158, filed Jan. 26, 2004, the disclosures of which are hereby incorporated by reference in the entirety for all purposes.

### FIELD OF THE INVENTION

The present invention relates to a device and system for removing fluid from a wound on a patient.

### BACKGROUND OF THE INVENTION

After a surgical procedure, body fluid may tend to collect near a wound created by the procedure. It is generally desirable to withdraw such body fluid from the wound site because doing so may accelerate wound healing and prevent a number of possible complications, such as hematomas, seromas and the like.

Evacuator systems for removing body fluid from a wound on a patient are known. Such systems typically include a drain, a conduit and a fluid container. The drain is usually inserted into the patient's wound before the site is surgically closed. Proper surgical closure of the wound site usually creates an essentially airtight seal for the drain within the wound site. The conduit is typically connected to the drain before the wound site is closed, and the conduit extends outside the patient's wound site. The other end of the conduit is normally attached to the fluid container. The fluid container is usually a plastic vessel having an internal reservoir, an entry port, to which the conduit is attached, and an exit port for expelling fluid collected in the internal reservoir.

In operation, vacuum is applied from the fluid container through the conduit to the drain. This draws body fluid, particularly blood, at or near the wound site through the drain, through the conduit and into the fluid container. Vacuum is often created from expansion (or decompression) of the fluid container after it has been compressed, or from an external vacuum source supplying the fluid container.

There are a number of problems with such evacuator systems, particularly related to clogging. One problem is that either the drain, the conduit, the entry port or exit port may clog. The drains in common use today, typically include a lumen (i.e., an internal passage in a drain or conduit), and know lumens have a diameter of approximately 3 millimeters or less. The size of the lumen for the conduit connected to the drain as well as the size of the passage in the entry port of the fluid container are essentially the same as that of the drain. Therefore, the conduit lumen and the entry port passage typically have diameters of approximately 3 millimeters or less. With such diameters, it is common for the drain, the conduit and/or the passage to clog with matter, such as clots, drawn from the wound site.

Another problem with such evacuator systems is that the diameter of the passage of the exit port is also typically 3 millimeters or less. Consequently, when it is desirable to expel the fluid from the fluid container, due to the narrow diameter of the exit port it may clog and create difficulties in draining and/or increase the time to drain the fluid container.

Yet another problem with such evacuator systems is their limited volumetric capacity. Typical evacuator systems today may be able to collect a body-fluid volume of less than 100 cubic centimeters. Consequently, the fluid containers of such evacuator systems must be more frequently drained than would be the case for larger-capacity fluid containers.

Still another problem with such evacuator systems is their use of valves, such as duck-bill-type valves, on the entry port passage through which fluid enters the internal reservoir of the fluid container. Such valves are structured to open and allow fluid to enter the reservoir of the fluid container, and to close and prevent fluid from being expelled back into the wound. Known designs, however, have such a small opening for allowing fluid to enter the reservoir that they tend to clog. Duck-bill valves typically have two members angled towards one another in a shape resembling a duck's bill and include an opening resembling a slit that faces inward towards the container reservoir. The bill essentially restricts fluid flow to one direction—into the fluid container's reservoir. When in use, such valves may become clogged, rendering the system ineffective until the clog is removed.

### SUMMARY OF THE INVENTION

In accordance with an embodiment of the invention, a device is disclosed comprising a container having an internal reservoir and a passage in communication with the internal reservoir. The passage is preferably cylindrical and has a diameter greater than 3 millimeters. A conduit in communication with the wound is connected to the passage. (In this context, “connected” simply means that the conduit and passage are in fluid communication.) Vacuum is applied through the passage and the conduit to draw fluid from the wound and into the internal reservoir of the container.

In accordance with another embodiment of the invention, a device is disclosed comprising a container having an internal reservoir and three or more passages in the container, wherein each passage is in communication with the internal reservoir. Either of two of the passages may be connected to a conduit and used to transfer vacuum from the reservoir through a conduit to a wound. The remaining passage is used for expelling fluid collected in the reservoir. Preferably the two passages that may be connected to a conduit are of different sizes so that one size conduit (such as a conduit having a lumen diameter of about 3 mm) may be used with one of the passages, and another size conduit (such as a conduit having a lumen diameter of about 6 mm) may be used with the other of the passages.

In accordance with still another embodiment of the invention, a device is disclosed comprising (1) a container having an internal reservoir and a passage in communication with the internal reservoir, and (2) a valve that prevents fluid from flowing from the reservoir through the passage, wherein the valve has an opening designed so that clogging is alleviated, and preferably has an opening approximately equal to the size of the passage.

Features and benefits of the invention will be set forth in part in the description which follows, and will in part be obvious from the description, or may be learned by practice of the invention.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the invention.

### BRIEF DESCRIPTION OF THE DRAWINGS

FIG. 1 is a block diagram of an evacuator system in accordance with the present invention.

FIG. 2 is a perspective diagram of a container that may be used as part of the evacuator system of FIG. 1, with the cover removed.

FIG. 3 is a perspective diagram of the cover for the container of FIG. 2.

FIG. 4A-4D are plan views of different covers that may be used with the container of FIG. 2.

FIG. 5A is a top, perspective view of a cover that may be used with the container of FIG. 2.

FIG. 5B is a bottom, perspective view of a cover that may be used with the container of FIG. 2.

### DESCRIPTION OF THE EMBODIMENTS

Reference will now be made to the exemplary embodiments of the invention, which are illustrated in the accompanying drawings.

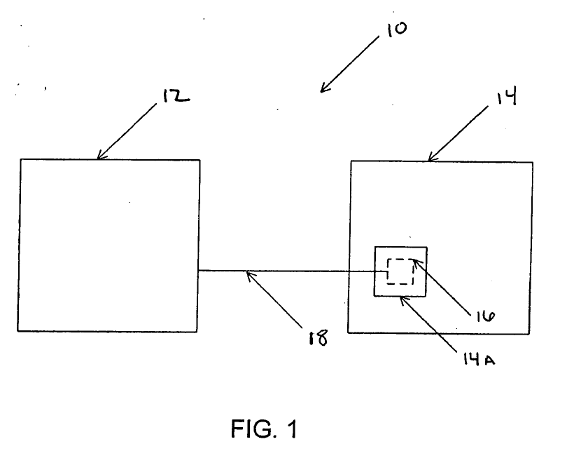

Referring to FIG. 1, an evacuator system 10 is shown for treatment of a patient 14 having a wound (or wound site) 14A. System 10 includes a fluid container 12, and may further include a drain 16 and a conduit 18. Drain 16 is shown in dashed lines to signify that it may be covered by surgical closure of wound site 14A, preferably, but not necessarily, creating an airtight seal for drain 16 within wound site 14A. Conduit 18 may be connected to drain 16 before wound site 14A is closed, so conduit 18 may extend outside wound site 14A and be connected to fluid container 12.

Drain 16 receives body fluid from patient 14. Any structure suitable for this purpose may be employed. In one embodiment, drain 16 may comprise a drain with a lumen having a diameter above 3 millimeters, such as the drain available from J.S. Vascular of Scottsdale, Ariz. and disclosed in copending U.S. Patent Provisional Application No. 60/476,663 filed on Jun. 5, 2003 and entitled “Improved Surgical Drains,” the disclosure of which is incorporated herein by reference.

Conduit 18 conveys body fluid received from drain 16 to fluid container 12. Any structure suitable for this purpose may be employed. In one embodiment, conduit 18 may comprise any conduit with a lumen having a diameter above 3 millimeters.

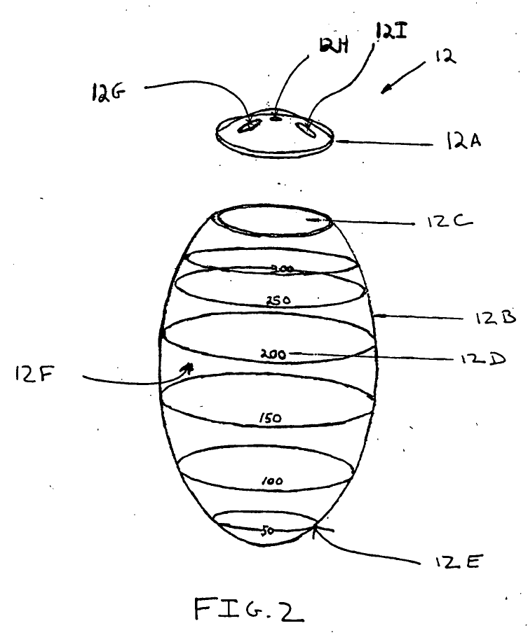

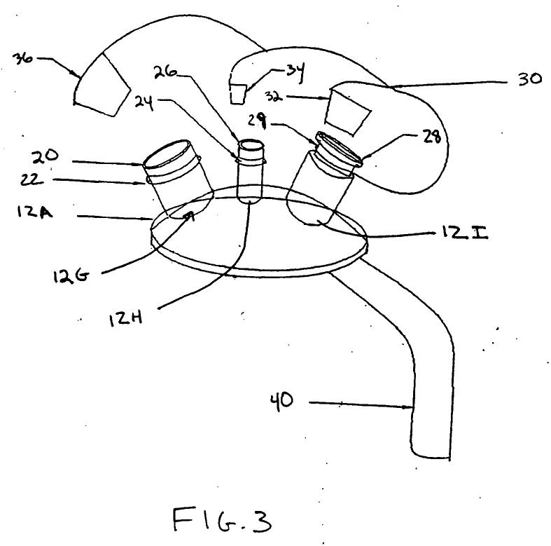

In one embodiment, fluid container 12 may include a container cover 12A and a container body 12B, which when assembled together define an internal reservoir 12F. Container cover 12A and container body 12B may be assembled in any suitable manner prior to use, such as by heat sealing during manufacturing or by cover 12A being threadingly received on body 12B. Fluid container 12 includes one or more passages, such as passages 12G-121, which as further described with respect to FIGS. 3-5, permit passage of fluid, such as body fluid from wound 14A into reservoir 12F or from reservoir 12F to another container when fluid container 12 is being emptied. Fluid container 12 may also include markings 12D to indicate the quantity of fluid that may be collected in fluid container 12. For example, as shown in FIG. 2, fluid container 12 may include markings 12D in 50 cubic-centimeter increments up to 300 cubic centimeters.

Vacuum is applied to draw body fluid from wound site 14A, via drain 16 and conduit 18 into internal reservoir 12F. Any structure or device suitable for this purpose may be employed. Moreover, the vacuum may be created from expansion (or decompression) of fluid container 12 after it has been compressed, and/or from an external vacuum source (not shown) that provides vacuum to fluid container 12.

Referring to FIG. 2, fluid container 12 may comprise a single, integral part or multiple assembled parts. Fluid container 12 may be manufactured using any compressible and resilient material. As a result, fluid container 12, may be compressed and then allowed to expand to create vacuum suitable to draw, or assist in drawing, fluid from wound 14A into internal reservoir 12F. When assembled, the uncompressed shape of fluid container 12 may generally resemble an ellipsoid, however, fluid container 12 may have any material property and any shape that is suitable for the purposes of evacuator system 10.

When in use, fluid container 12 may lie on its side or be configured to stand upright, as shown in FIG. 2. In the latter case, fluid container 12 may include a variation from the embodiment shown in FIG. 2, namely having a lower section, such as section 12E, removed and sealed to create a flat surface to be supported. Such a variation may be completed during manufacture of fluid container 12. Any suitable surface may then be used to support fluid container 12 on an end in an upright manner. Fluid container 12 may also include any structure suitable for attaching fluid container 12 to any article, e.g., attaching fluid container 12 to an article such as clothing or bedding near patient 14. A shown in FIG. 3, a lanyard 40 and an attaching pin (not shown) may serve this purpose.

A variation of container cover 12A is shown in FIG. 3. Extending from and in communication with passages 12G, 12H and 121 may be respective ports 20, 26 and 28. Port 26 may serve as a fluid input to reservoir 12F and may have a diameter corresponding to its respective passage 12H that accommodates a first type of conduit for a first drain 16, such as a drain with a lumen diameter of 3 millimeters or less. Port 20 may serve as a fluid input to reservoir 12F and may have a diameter corresponding to its respective passage 12G that accommodates a second type of conduit for a second drain 16, such as a drain with a lumen diameter greater than 3 millimeters.

Port 28 may serve as a fluid output from fluid container 12 and may have a diameter corresponding to its respective passage 121, e.g., greater than 3 millimeters, to improve the draining efficiency of fluid container 12. In one embodiment of fluid container 12, the respective diameters passages 12H, 12G and 121 are 3 millimeters, 6 millimeters and 15 millimeters.

Ports 20 and 26 may include respective retainers 22 and 24, such as chevrons, to retain respective conduits (not shown), by pressing a respective conduit over the port. However, any structure for placing a conduit in fluid communication with internal reservoir 12F may be utilized.

Port 28 may include a neck 29 to receive a band 30 for retaining stoppers 36, 34 and 32 for their respective ports 20, 26 and 28. Any material may be used for lanyard 40, so that it may be reused to attach fluid container 12 to an article, such as an article of clothing or bedding near patient 14. Container cover 12A may also include one or more valves to prevent backflow through any input passages, such as passages 12G and 12H, back to patient 14.

In operation, one of the fluid input ports 20 or 26 may be blocked with its respective stopper 36 or 34. The other input port 20 or 26 may be connected to a conduit 18 that may be attached to a drain 16 in wound site 14A. With the output port stopper 32 removed, fluid container 12 may be compressed to force fluid, e.g., air and/or body fluid, out of internal reservoir 12F through output port 28. The output port stopper 30 may then be reinserted. Fluid container 12 may then, due its resiliency, begin expanding to an uncompressed state, thus creating suction or vacuum via conduit 18 and drain 16 to remove body fluid from wound site 14A.

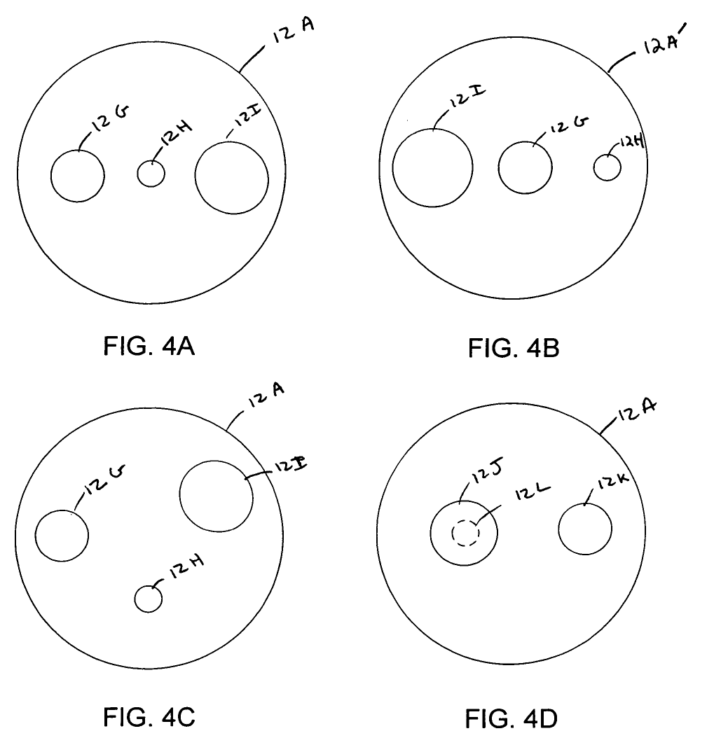

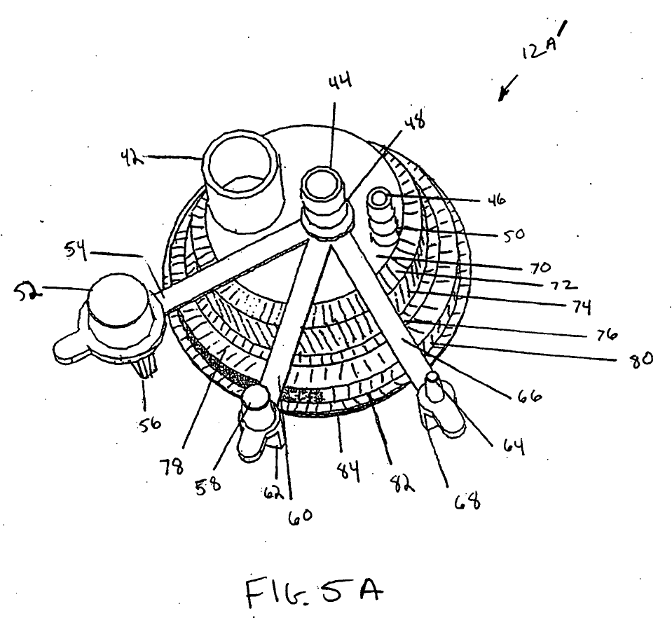

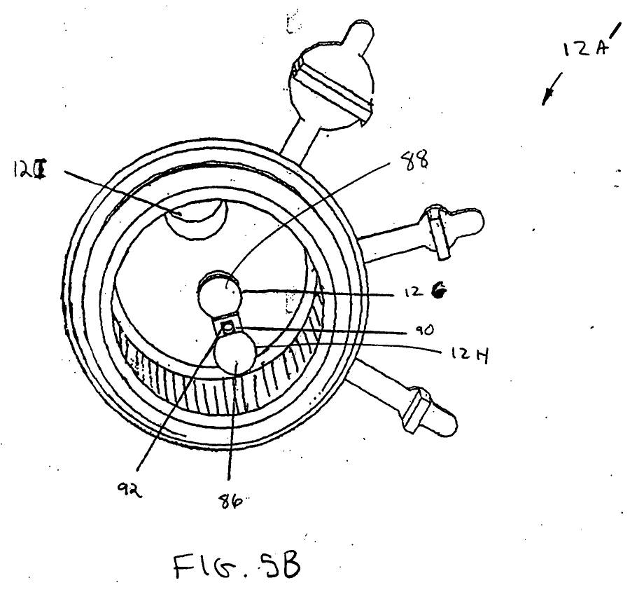

Referring to FIGS. 4A-4D, different configurations are shown in plan view of the passages in container cover 12A, understanding that any other number or configuration of passages is permissible. FIG. 4A depicts a plan view of passages 12G-121 in container cover 12A from FIGS. 2 and 3. FIG. 4B depicts a plan view of passages 12G-121 in container cover 12A′ from FIGS. 5A-5B. FIG. 4C depicts a plan view of passages 12G-121 not aligned in single-row fashion, but in another arrangement, such as triangular.

Regarding FIGS. 4A-4C, passage 12G may comprise a fluid inlet of diameter greater than 3 millimeters, e.g., 6 millimeters; passage 12H may comprise a fluid inlet of diameter 3 millimeters or less, e.g., 3 millimeters; and passage 121 may comprise a fluid outlet of diameter greater than 3 millimeters, e.g. 15 millimeters.

In FIG. 4D, container cover 12A may include two or more passages, such as passages 12J and 12K. Passage 12J may comprise a fluid inlet of diameter greater than 3 millimeters, e.g., 6 millimeters; and passage 12K may comprise a fluid outlet of diameter greater than 3 millimeters, e.g. 15 millimeters. Passage 12 L, shown in broken lines, represents an adaptor permitting fluid inlet passage 12J, which may have a diameter greater than 3 millimeters to be selectively reduced to 3 millimeters or less in diameter. Any conventional adaptor for this purpose may be utilized.

A variation of container cover 12A is shown in FIGS. 5A-5B. Extending from and in communication with passages 121, 12G and 12H are respective ports 42, 44 and 46. Port 46 may serve as a fluid input to reservoir 12F and has a diameter corresponding to its respective passage 12H that accommodates a first type of conduit for a first drain 16, such as a drain with a lumen diameter of 3 millimeters or less. Port 44 may serve as a fluid input to reservoir 12F and has a diameter corresponding to its respective passage 12G that accommodates a second type of conduit drain 18, such as a drain with a lumen diameter greater than 3 millimeters.

Port 42 may serve as an output port so that fluid from fluid container 12 may be expelled there through. Port 42 has a diameter corresponding to its respective passage 121, e.g., greater than 3 millimeters, to improve the draining efficiency of fluid container 12.

In one embodiment of fluid container 12, the respective lumen diameters for passages 121, 12H and 12G are 15 millimeters, 3 millimeters and 6 millimeters.

Ports 44 and 46 may include respective retainers 48 and 50, such as chevrons, to selectively retain conduits (not shown). Port 44 may retain bands 54, 60 and 66 for retaining stoppers 52, 58 and 64 for their respective ports 42, 44 and 46. Grippers 56, 62 and 68 may be provided to help handling respective stoppers 52, 58 and 64. A lanyard may be similarly employed, as previously described, to attach fluid container 12 to an article, such as an article of clothing or bedding near patient 14.

While container cover 12A shown in FIGS. 2-3 may have a generally smooth, dome-like shape, the container cover may be of any suitable shape. For example, container cover 12A′ in FIGS. 5A-5B may have a more tiered, dome-like shape, and the assembled fluid container 12 may have the general shape of an ellipsoid, regardless of whether container cover 12A or 12A′ is used. However, fluid container 12 could be any shape or size suitable to collect fluid from a wound in any of the manners described herein. The more-tiered structure of container cover 12A′ may include, as shown from top to bottom in FIG. 5A, a flat top surface 70, a beveled surface 72, a circular sidewall 74, a flared portion 76, a support rim 78, a circular sidewall 80, a flange 82 and a rim 84.

The bottom view of fluid container 12A′ in FIG. 5B shows a pair of valves that may restrict fluid flow to one direction through inlet passages 12H and 12G, namely into the fluid container 12. Consequently, backflow of body fluid to patient 14 may be prevented. The valves may comprise flapper-type valves. For inlet passage 12H, a first section of material or flapper 86 may be provided to restrict flow through inlet passage 12H when flapper 86 seals inlet passage 12H. Flapper 86 may be biased to seal inlet passage 12H. Any suitable material and biasing may be employed. Moreover, the diameter of flapper 86 may be greater than or equal to the diameter of its corresponding inlet passage 12H, so that a proper seal may be effected.

When appropriate vacuum is provided within reservoir 12F and passages 12G and 121 are sealed with their respective stoppers 58 and 52. The differential pressure across flapper 86 will be transferred into conduit 18 and drain 16 and will cause fluid flow into reservoir 12F. When vacuum is reduced enough, the biasing of flapper 86 may seal inlet passage 12H. Moreover, if positive pressure is applied to internal reservoir 12F the pressure pushes flapper 86 shut and helps to prevent fluid from moving from reservoir 12F through passage 12H.

For inlet passage 12G, a second section of material or flapper 88 may be provided to restrict flow through inlet passage 12G when flapper 88 seals inlet passage 12G. Flapper 88 preferably operates similarly to flapper 86. Flapper 88 may be biased to seal inlet passage 12G. Any suitable material and biasing may be employed. Moreover, the diameter of flapper 88 may be greater than or equal to the diameter of its corresponding inlet passage 12G, so that a proper seal may be effected.

A section of material 90 may interconnect flappers 86 and 88. A post 92 may be inserted through an aperture in interconnecting material section 90 to prevent flappers 86 and 88 from rotating off-axis with respect to their respective passages 12H and 12G. For example, post 92 may have an angled cross section, e.g., square, which may prevent interconnecting material section 90 and flappers 86 and 88 from rotating off-axis. Other designs may be utilized to prevent interconnecting material section 90 and flappers 86 and 88 from rotating off-axis.

In operation, one of the fluid input ports 44 or 46 is blocked with its respective stopper 58 or 64. The other input port 44 or 46 is connected to a conduit 18 that may be attached to a drain 16 in wound site 14A. With the output port stopper 52 removed, fluid container 12 may be compressed to force air and/or body fluid, out of internal reservoir 12F through output port 42. The output port stopper 52 may then be reinserted into port 42. Fluid container 12 may then, due its resiliency, begin expanding to an uncompressed state, thus creating suction or vacuum through unblocked fluid port 44 or 46 and through conduit 18 and drain 16 in fluid communication with port 44 or 46 in order to remove body fluid from wound site 14A and transfer it to internal reservoir 12F.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A device for drawing fluid from a wound, the device comprising:
a container having an internal reservoir from which vacuum may be applied through a conduit to a wound; and
a passage in the container and to which the conduit is connected, the passage having a diameter greater than 3 millimeters and for transferring fluid from the conduit to the internal reservoir.

2. The device of claim 1 further comprising the conduit.

3. The device of claim 1 wherein the container is compressible and resilient.

4. The device of claim 1 wherein vacuum is created by decompression of the container after the container has been compressed.

5. The device of claim 1 wherein the container has an uncompressed shape of an ellipsoid.

6. The device of claim 1 wherein the container is configured to be supported on a surface.

7. The device of claim 6 wherein the configuration to support the container on the surface comprises a flat surface on an end of the container.

8. The device of claim 1 wherein the container is configured to be attached to an article.

9. The device of claim 8 wherein the article is near a patient with the wound.

10. The device of claim 1 wherein the internal reservoir has a volume greater than 100 cubic centimeters.

11. The device of claim 1 wherein the container includes markings to indicate a plurality of volumetric quantities of fluid that may be collected in the container.

12. The device of claim 1 further comprising means for retaining the conduit to the container.

13. The device of claim 1 further comprising valve means for blocking fluid flow from the internal reservoir through the passage.

14. The device of claim 1 further comprising a second passage for connecting to a second conduit and for transferring fluid from the second conduit to the internal reservoir.

15. The device of claim 13 wherein the vacuum may be applied through the second passage to the wound.

16. The device of claim 13 wherein the vacuum may be applied through only one of the passages at a time.

17. The device of claim 13 further comprising the second conduit.

18. The device of claim 13 wherein the second passage is greater than or equal to 3 millimeters in diameter.

19. The device of claim 17 wherein the second passage is 3 millimeters in diameter.

20. The device of claim 13 further comprising means for retaining the second conduit to the container.

21. The device of claim 14 further comprising means for blocking fluid flow through the second passage.

22. The device of claim 13 further comprising a third passage in the container.

23. The device of claim 22 wherein the third passage is greater than or equal to 3 millimeters in diameter.

24. The device of claim 1 further comprising a valve that restricts fluid flow to one direction through the passage, the valve not comprising a duck-bill-type valve.

25. The device of claim 24 wherein the valve comprises a flapper-type valve.

26. The device of claim 14 further comprising a valve that restricts fluid flow to one direction through the second passage, the valve not comprising a duck-bill-type valve.

27. The device of claim 26 wherein the valve comprises a flapper-type valve.

28. A device for draining fluid from a wound, the device comprising:
a container having an internal reservoir from which vacuum may be applied to a wound; and
three or more passages in the container, one or more of the three or more passages being for connecting to a conduit so that vacuum may be applied to the wound, and one or more of the three or more passages being for expelling fluid collected in the reservoir.

29. The device of claim 27 wherein one or more of the three or more passages is greater than or equal to 3 millimeters in diameter.

30. The device of claim 27 wherein one of the three or more passages is 3 millimeters in diameter, another of the three or more passages is 6 millimeters in diameter and another of the three or more passages is 15 millimeters in diameter.

31. The device of claim 27 wherein two or more of the three or more passages are fluid-flow inlets from the wound to the internal reservoir.

32. The device of claim 27 wherein one or more of the three or more passages are fluid-flow outlets for one or more of draining and evacuating the internal reservoir.

33. The device of claim 27 further comprising the one or more conduits.

34. The device of claim 27 wherein the container is compressible and resilient.

35. The device of claim 27 wherein the vacuum is created by decompression of a compressed container.

36. The device of claim 27 wherein the container is configured to be supported on a surface.

37. The device of claim 35 wherein the configuration to support the container on the surface comprises a flat surface on an end of the container.

38. The device of claim 27 wherein the container is configured to be attached to an article.

39. The device of claim 37 wherein the article is near a patient with the wound.

40. The device of claim 28 wherein the internal reservoir has a volume greater than 100 cubic centimeters.

41. The device of claim 27 wherein the container includes markings to indicate a plurality of volumetric quantities of fluid that may be collected in the container.

42. The device of claim 27 further comprising means for retaining the one or more conduits to the container.

43. The device of claim 27 further comprising means for blocking fluid flow through the three or more passages.

44. The device of claim 27 further comprising a valve that restricts fluid flow to one direction through one of the three or more passages, the valve not comprising a duck-bill-type valve.

45. The device of claim 44 wherein the valve comprises a flapper-type valve.

46. The device of claim 44 further comprising a second valve that restricts fluid flow to one direction through a second one of the three or more passages, the second valve not comprising a duck-bill-type valve.

47. The device of claim 46 wherein the second valve comprises a flapper-type valve.

48. A device for draining fluid from a wound, the device comprising:
a container having an internal reservoir and a passage in communication with the internal, wherein vacuum can be applied through the passage to draw fluid from a wound and into the reservoir; and
a valve that prevents backflow of the fluid through the passage, the valve not comprising a duck-bill-type valve.

49. The device of claim 48 wherein the valve comprises a flapper-type valve.

50. The device of claim 2 further comprising a drain for insertion into the wound and for connection to the conduit.

51. The device of claim 32 further comprising a drain for insertion into the wound and for connection to one or more of the conduits.

52. The device of claim 48 further comprising:
a surgical drain for insertion into the wound; and
a conduit for connection at one end to the surgical drain and for connection at another end to the container.
